(19) United States
(12) Patent Application Publication
(10) Pub. No.: US 2026/0324957 A1
(43) Pub. Date: Oct. 22, 2026

(54) ARTICULATION JOINTS AND DISTAL TIPS FOR MEDICAL DEVICES AND SYSTEMS

(71) Applicant: Boston Scientific Scimed, Inc., Maple Grove, MN (US)

(72) Inventor: Sean POWELL, Holden, MA (US)

(73) Assignee: Boston Scientific Scimed, Inc., Maple Grove, MN (US)

(21) Appl. No.: 19/467,983

(22) Filed: Feb. 3, 2026

Related U.S. Application Data
(60) Provisional application No. 63/753,514, filed on Feb. 4, 2025.

(51) Int. Cl. A61B 1/008; A61B 1/00; A61B 1/018

(52) U.S. Cl. CPC A61B 1/008; A61B 1/00114; A61B 1/018

(57) ABSTRACT

An articulation section for the medical device includes a plurality of intermediate links that defines a first channel, a distalmost link that defines a second channel in communication with the first channel and a third channel in communication with the first channel, and a proximalmost link that defines a lumen in communication with the first channel. The first channel includes a first opening on a radially outer side of each of the intermediate links. At least a portion of the second channel has a second opening on a radially outer side of the distalmost link, and at least a portion of the third channel has a third opening on a radially outer side of the distalmost link. The lumen is fully enclosed on all radially outer sides of the lumen.

### CROSS-REFERENCE TO RELATED APPLICATION(S)

This application claims the benefit of priority to U.S. Provisional Application No. 63/753,514, filed on February 4, 2025, which is incorporated by reference herein in its entirety.

### FIELD OF INVENTION

The disclosure relates generally to medical devices, and more particularly to articulation joints and distal tips for endoscopic medical devices.

### BACKGROUND

Endoscopic medical devices generally include a handle, a flexible shaft, a working distal tip, and a flexible steerable shaft joining the working tip and the handle. Control elements, working channel(s), electronic elements, fluidic tubes, or other elements extend through the shaft, from the handle to the distal tip. The flexible steerable shaft often incorporates an articulation joint to allow navigation through anatomical structures during a medical procedure. A need exists for improved articulation joints for endoscopic devices.

### SUMMARY

Examples of this disclosure relate to, among other things, medical systems, devices, and methods. For example, examples of this disclosure relate to medical devices and systems that utilize articulation joints having a plurality of links.

According to one aspect, the disclosure provides an articulation section for a medical device including a plurality of intermediate links defining a first channel, a distalmost link defining a second channel in communication with the first channel and a third channel in communication with the first channel, and a proximalmost link defining a lumen in communication with the first channel. The first channel has a first opening on a radially outer side of each of the plurality of intermediate links. At least a portion of the second channel has a second opening on a radially outer side of the distalmost link, and at least a portion of the third channel has a third opening on a radially outer side of the distalmost link. The lumen is fully enclosed on all radially outer sides of the lumen.

According to some aspects, the articulation section may include a tube positioned within the first channel, the second channel, and the lumen. The articulation section may include a cable or a wire positioned within the first channel, the third channel, and the lumen.

According to some aspects, each of the plurality of intermediate links may include a wall defining the first channel. The wall may include an apex, two tapered surfaces extending from the apex, and two side surfaces extending from the two tapered surfaces. Radially outer ends of the two side surfaces may define a gap that defines the first opening. The articulation section may include a wire positioned adjacent to the apex. The first opening may extend along an entire length of a portion of the articulation section having the plurality of intermediate links. The articulation section may include at least one articulation wire extending through a lumen of each extending through the plurality of intermediate links. The first channel may have cross-sectional shape that is a pentagon or a rounded pentagon.

According to some aspects, only a portion of a longitudinal length of the second channel may have the second opening. Only a proximal portion of the second channel may have the second opening.

According to some aspects, an entire longitudinal length of the third channel may have the third opening. The third channel may have a distal portion that is wider than a proximal portion of the third channel, in which the distal portion of the third channel includes an imager.

According to some aspects, a cross-sectional shape of the lumen may be non-circular. The cross-sectional shape of the lumen may be a diamond or a rounded diamond.

In another aspect, the disclosure provides an articulation section for a medical device, the articulation section comprising: a plurality of links defining a channel. The channel has an opening on a radially outer side of each of the plurality of links, wherein a wall of each link of the plurality of links defines the channel, wherein the wall includes an apex, two tapered surfaces, and two side surfaces, such that the channel has a pentagonal or a rounded pentagonal shape. The articulation section also comprises a cable or a wire positioned within the channel near the apex, and a working channel positioned within the channel between the cable or the wire and the opening.

According to some aspects, the plurality of links may be a plurality of intermediate links, wherein the articulation section may further comprise a distalmost link having a first channel and a second channel, wherein the working channel may extend through the first channel of the distalmost link, and wherein the cable or the wire may extend through the second channel of the distalmost link. The channel may be a first channel, wherein the articulation section may further comprise a proximalmost link having a lumen that is enclosed on all radial outer sides of the lumen, and wherein (a) the working channel and (b) the cable or the wire may extend through the lumen.

According to yet another aspect, the disclosure provides an articulation section for a medical device, the articulation section comprising: a plurality of intermediate links defining a first channel, a distalmost link defining a second channel in communication with the first channel and a third channel in communication with the first channel, a working channel extending through the first channel and the second channel; and a cable or a wire extending through the first channel and the third channel. The first channel has a first opening on a radially outer side of each of the plurality of intermediate links. At least a portion of the second channel has a second opening on a radially outer side of the distalmost link. At least a portion of the third channel has a third opening on a radially outer side of the distalmost link.

According to some aspects, a distal end of the cable or the wire is coupled to an imager.

The foregoing general description of the illustrative embodiments and the following detailed description thereof are merely exemplary aspects of the teachings of this disclosure and are not restrictive.

### BRIEF DESCRIPTION OF FIGURES

The accompanying drawings, which are incorporated in and constitute a part of this specification, illustrate exemplary aspects of the disclosure and, together with the description, serve to explain the principles of the disclosure.

FIG. 1 depicts a medical system, according to aspects of the disclosure.

FIG. 2A depicts a partially transparent perspective view of a distal portion of a medical device.

FIG. 2B depicts a cross-sectional view of the distal portion of FIG. 2A.

FIG. 3 depicts a perspective view of a link of articulation joint and distal tip of the distal portion of FIG. 2A, according to aspects of the disclosure.

FIG. 4 depicts another cross-sectional view of the distal portion of FIG. 2A, according to aspects of the disclosure.

FIG. 5 depicts a further cross-sectional view of the distal portion of FIG. 2A, according to aspects of the disclosure.

FIGS. 6A and 6B depict perspective views of a portion of the distal tip of the distal portion of FIGS. 2A and 2B, according to aspects of the disclosure.

FIGS. 7A depicts a perspective view of an adapter of the articulation joint of FIG. 3.

FIG. 7B depicts a proximally facing view of a distal end of the adapter of FIG. 7A.

### DETAILED DESCRIPTION

Examples of the disclosure include devices, systems, and methods for articulation joints and distal tips. An endoscopic system/device may include a handle assembly and a shaft. The shaft may extend distally from the handle to a distal tip and may include an articulation section having an articulation joint. For example, the articulation joint may have a plurality of middle links defining a channel therethrough. The channel may have an opening on an outer periphery of the articulation joint. A distalmost link of the articulation joint may form a core of a distal tip of the endoscopic system/device. The channel of more proximal portions of the articulation joint may separate into two channels within the distalmost link. Each of the two channels may have an opening on the outer periphery of the articulation joint. A proximalmost link of the articulation joint may form an adapter for connecting with a distal end of a tubular member of the shaft. The proximalmost link may define a channel that is closed on all sides. One or more elements may extend through the channel of the proximalmost link, into the channel of the middle links, and into one of two channels of the distalmost link.

As used herein, the term "distal" refers to a portion farthest away from a user when introducing a device into a patient and the term "proximal" refers to a portion closest to the user when placing the device into the subject. Arrows labeled “P” and “D” in the figures label proximal and distal directions, respectively. The terms "comprises," "comprising," “includes,” “including,” “has,” “having,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term "exemplary" is used in the sense of "example," rather than "ideal." As used herein, the terms "about," "substantially," and "approximately," and the like indicate a range of values within +/- 10% of a stated value.

Examples of the disclosure may relate to system, devices, and methods for performing various medical procedures and/or treating portions of the biliary duct, large intestine, small intestine, cecum, esophagus, any other portion of the gastrointestinal tract, and/or any other suitable patient anatomy (collectively referred to herein as a "target site"). The devices disclosed herein may be entirely disposable, entirely reusable, or may include a combination of disposable and reusable devices. Reference will now be made in detail to examples of the disclosure described above and illustrated in the accompanying drawings. Wherever possible, the same reference numbers will be used throughout the drawings to refer to the same or like parts.

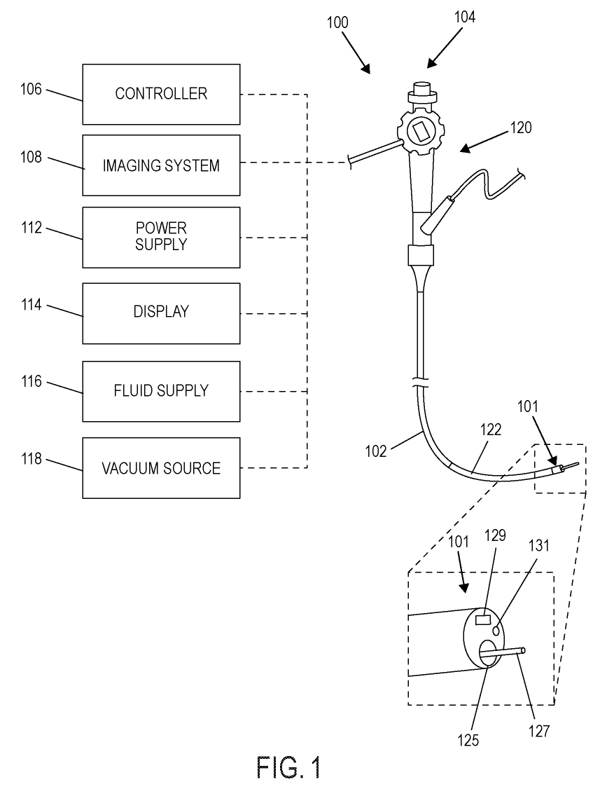

An exemplary endoscopy system 100 is shown in FIG. 1. Endoscopy system 100 may include an endoscope 104. Endoscope 104 may include a handle assembly 120 and a flexible tubular shaft 102. The flexibility of shaft 102 may be sufficient to allow shaft 102 to bend, to facilitate navigation of shaft 102 through a subject's tortuous anatomical passages. Shaft 102 may terminate at a distal tip 101. Shaft 102 may include an articulation section 122 for deflecting distal tip 101 in up, down, left, and/or right directions. In one example, articulation section 122 may provide for full retroflexion (e.g., rotation of distal tip 101 through an arc of 180 degrees) or only partial retroflexion (e.g., rotation of distal tip 101 through an arc of less than 180 degrees). Endoscope 104 also may include one or more lumens extending therethrough, and one or more openings in communication with the one or more lumens. For example, the one or more lumens may extend through handle assembly 120 and shaft 102, and the one or more openings may be on handle assembly 120 and distal tip 101. Endoscope 104 may be any suitable device for insertion into a patient's body, such as, e.g., an endoscope, a gastroscope, a ureteroscope, a nephroscope, a colonoscope, a hysteroscope, a ureteroscope, a bronchoscope, a cystoscope, a duodenoscope, a sheath, or a catheter.

One or more auxiliary devices may be operatively coupled to endoscope 104. Exemplary auxiliary devices may include a controller 106, an imaging system 108, a power supply 112, a display 114, a fluid supply assembly 116, and/or a vacuum source 118, each of which is briefly described below. Controller 106 may include, for example, any electronic device capable of receiving, storing, processing, generating, and/or transmitting data according to instructions given by one or more programs. Controller 106 may be operatively coupled to, or part of, one or more of endoscope 104 and the other auxiliary devices, to control one or more aspects of their operation. Power supply 112 may include any suitable power source, and associated connectors (e.g., electrically-conductive wires), for supplying electronic components in the auxiliary devices and endoscope 104 with electrical power. Fluid supply assembly 116 may include a reservoir, a medical irrigation bag, a pump, and any suitable connectors (e.g., tubing for fluidly coupling fluid supply assembly 116 and endoscope 104). The pump may supply a flow of pressurized fluid to one or more of the lumens in endoscope 104, and the pressurized fluid flow may be emitted from distal tip 101 and/or used to inflate expandable components present at distal tip 101. Vacuum source 118 may provide suction or vacuum pressure to one or more lumens of the endoscope, and thereby provide a suction force to draw material toward and/or into endoscope 104, and/or to deflate expandable components.

Imaging system 108 may include imaging electronics to, for example, process signals received from an image sensor in endoscope 104, send signals for controlling the image sensor, adjust illumination levels of areas being viewed by the image sensor, and/or facilitate the display of image sensor data on display 114.

Distal tip 101 may include one or more imagers 129 and one or more illuminators 131, shown in the magnified view of distal tip 101 in FIG. 1. One or more imagers 129 may include a charge-coupled device image sensor, a complementary metal-oxide image semiconductor, or the like coupled to a cable or wire running through the shaft 102 of endoscope 104. Alternatively, imager 129 may include fiber optic elements. One or more illuminators 131 may include light emitting diodes (LEDs), fiber optic elements, or the like.

A tool 127 may be inserted into a working channel or a lumen 125 of endoscope 104 and may exit out of the distal end of lumen 125. Tool 127 may include, for example, a guidewire, cutting or grasping forceps, a biopsy device, a snare loop, an injection needle, a cutting blade, scissors, a retractable basket, a retrieval device, an ablation and/or electrophysiology catheter, a stent placement device, a surgical stapling device, a balloon catheter, a laser-emitting device, and/or any other suitable therapeutic or diagnostic instrument. As shown in the magnified view of distal tip 101, tool 127 has a smaller circumference about its longitudinal axis compared to the circumference about the longitudinal axis of lumen 125, and may include a smaller cross-sectional diameter as compared to the diameter of lumen 125.

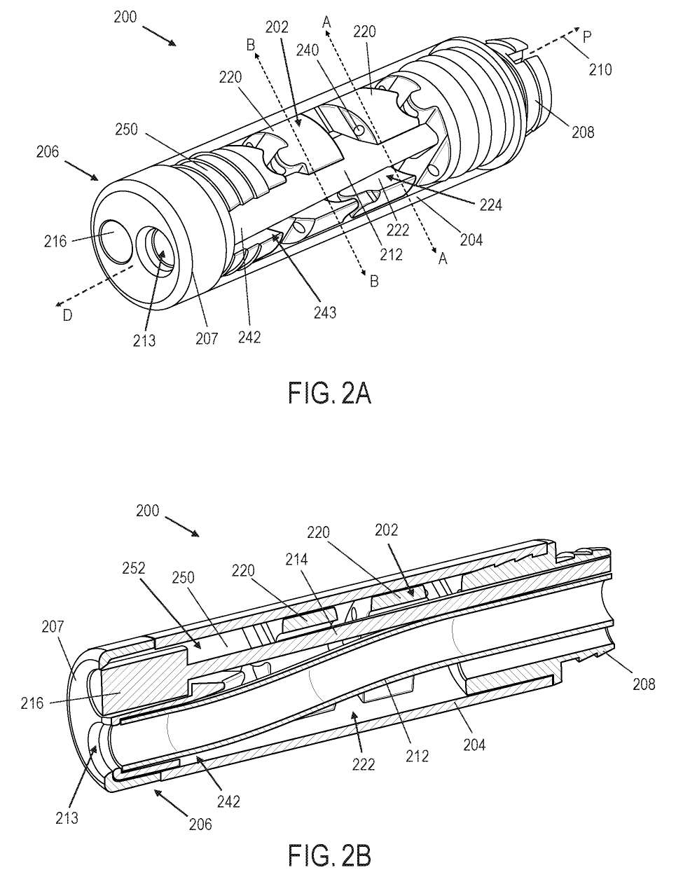

FIGS. 2A and 2B illustrate an articulation section 200, including an articulation joint 202 arranged along a central longitudinal axis 210 of articulation section 200. FIG. 2A shows a perspective view of articulation section 200, and FIG. 2B shows a cross-sectional view taken parallel to central longitudinal axis 210. Articulation section 200 may have any of the properties of articulation section 122 and may form a distal portion of shaft 102. A sheath 204 may cover articulation joint 202 (and, although not shown in FIGS. 2A-2B, more proximal portions of shaft 102). In FIG. 2A, sheath 204 is shown as transparent to reveal aspects of articulation section 200 internal to sheath 204. Sheath 204 may protect components of articulation joint 202 from a surrounding environment while allowing flexibility during medical procedures. Sheath 204 may be composed of a flexible and/or durable material, such as silicone, polyurethane, or a polymer, and may have varying thickness along its length to provide different degrees of flexibility and protection. Articulation section 200 may terminate in a distal tip 206, having any of the properties of distal tip 101.

Articulation joint 202 may allow for controlled bending of articulation section 200 during a medical procedure. For example, articulation section 200 may be inserted into a target site within patient anatomy and controlled via handle assembly 120, such that articulation section 200 may bend in one or more directions relative to central longitudinal axis 210 in response to actuation of an actuator (e.g., knob or lever) of handle assembly 120. This may be useful to control position and orientation of distal tip 206 relative to patient anatomy during the procedure. For example, distal tip 206 may include an imager 216 (having any of the properties of imager 129) and a distalmost opening 213 of a tube 212, which may function as a working channel or as a conduit for fluid (e.g., air or water) delivery. Although distal tip 206 is shown as having one opening 213, and articulation section 200 is shown as having one tube 212, any number of tubes 212 and openings 213 may be present. Although distal tip 206 is not shown as having any illuminators, such elements may be built into imager 216 or may be separate from imager 216 and disposed within distal tip 206 (e.g., as shown in FIG. 1).

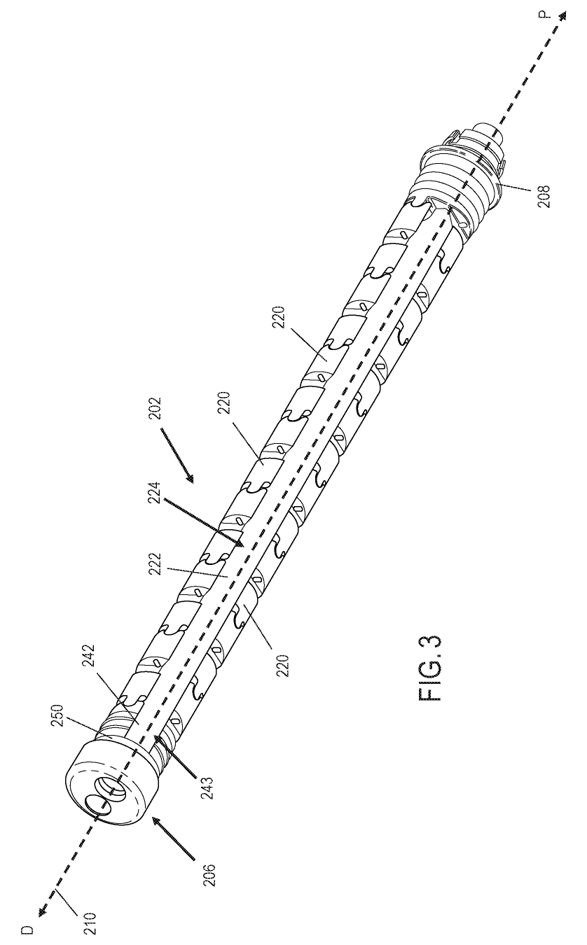

As shown in FIGS. 2A and 3, articulation joint 202 may include a plurality of links 220, for example arranged in a linear configuration along central longitudinal axis 210. Links 220 may be intermediate links along a longitudinally middle portion of articulation joint 202. Links 220 may be configured to allow controlled bending of articulation joint 202, providing flexibility to articulation section 200. In some implementations, each link 220 may be shaped or sized to facilitate interlocking with one or more adjacent links 220, for example to allow for articulation yet maintain structural integrity of articulation section 200 or to provide for a desired bending radius. Links 220 may include features such as protrusions, indentations, or beveled edges that guide and limit range of motion between adjacent links 220. A number of links 220 shown in FIGS. 2A and 2B is for ease of illustration only—articulation joint 202 may include more links 220, as shown in FIG. 3. A number of links 220 may be chosen to provide a desired bending profile of articulation joint 202.

A core 250 of distal tip 206 may be at a distal end of articulation joint 202. In some examples, core 250 may be a distalmost link of articulation joint 202 and may be hingedly coupled to a distalmost of links 220, as shown in FIGS. 2A and 3. For example, a distal end of core 250 may include recesses for cooperating with protrusions of a proximal end of an adjacent link 220 so that core 250 is pivotable with respect to the adjacent link 220. A cap 207 may be positioned over core 250 to form distal tip 206. In some aspects, cap 207 may be distal to sheath 204.

An adapter 208 for coupling articulation joint 202 to a distal end of tubular member of shaft 102 may be disposed at a proximal end of articulation joint 202. In some examples, adapter 208 may form a proximalmost link of articulation joint 202. Adapter 208 may be hingedly coupled to a proximalmost of links 220. For example, as shown in FIGS. 2A and 3, adapter 208 may include one or more protrusions at a distal end of adapter 208 for cooperating with protrusions of an adjacent link 220 so that adapter 208 is pivotable with respect to the adjacent link 220.

As shown particularly in FIGS. 2A, 3, and 4, links 220 may collectively define a channel 222 that extends along a length of articulation joint 202 defined by links 220. Channel 222 may be shaped and sized to accommodate various components, such as tube 212 or a cable 214 (or wire or plurality of cables or wires, such as a bundle of cables or wires) electronically coupled to imager 216. Cable 214 may provide power and/or electrical signals to or from imager 216. Tube 212 and cable 214 may extend from handle assembly 120 to distal tip 206. Although one tube 212 and one cable 214 are shown, multiple of each element may extend through channel 222. Further details of channel 222 are described below.

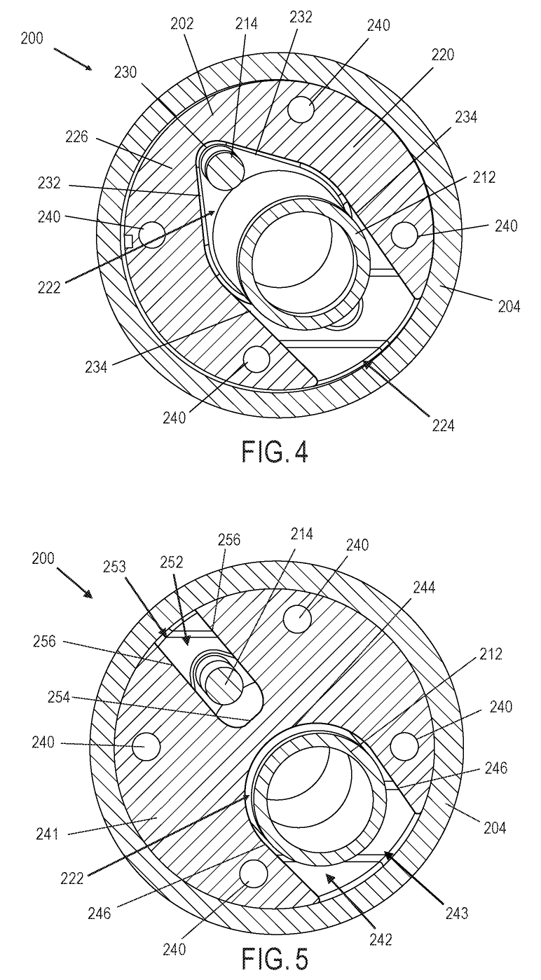

As shown particularly in FIGS. 2A and 4, links 220 may also define one or more lumens 240 (e.g., two or four lumens 240), for example arranged to house or accommodate one or more articulation wires or other components therein. In some examples, lumens 240 may have a circular cross-sectional shape or a non-circular cross-sectional shape, such as oval, square, triangle or other shape. Lumens 240 may be positioned to distribute forces during articulation and to reduce stress on articulation wires or links 220. For example, two lumens 240 may allow for two-way steering, while four lumens 240 may facilitate four-way steering. Articulation wires extending through lumens 240 may transmit forces from handle assembly 120 to articulation section 200, thereby allowing articulation of articulation joint 202.

Returning to FIGS. 2A, 3 and 4, channel 222 may have an opening 224 that is open on a radially outer surface of links 220. In other words, channel 222 may have a trench shape, extending inward into links 220 from opening 224. Thus, along an entire length of channel 222, channel 222 may be in communication with an area radially outside of links 220. Channel 222 and opening 224 may extend along an entire axial length of a portion of articulation joint 202 defined by links 220 (i.e., an entire length of articulation joint 202 that is distal to adapter 208 and proximal of core 250). As compared with a closed channel having walls of links fully radially surrounding the closed channel, channel 222 offers a larger volume or cross-sectional area to accommodate elements (e.g., tube 212 and cable 214).

FIG. 4 illustrates a cross-sectional view of articulation section 200, along a plane having line B-B, shown in FIG. 2A. In other words, FIG. 4 illustrates a cross-section of articulation section 200 that is taken perpendicularly to central longitudinal axis 210 and through one of links 220. Each of links 220 may have one or more walls 226, which define a structure of a respective link 220. Wall 226 may define channel 222 and lumens 240. For example, edges of wall 226 may define boundaries of channel 222 and boundaries of lumens 240.

A portion of wall 226 defining channel 222 has an apex 230, which may have a curved shape. For example, a curved shape of apex 230 may have a radius of curvature that is approximately the same as a radius of curvature of cable 214, so that cable 214 rests against apex 230. Apex 230 may be close to a side of link 220 that is opposite opening 224. For example, where articulation joint 202 includes four lumens 240 (as shown in FIG. 4), a line drawn between centers of two of lumens 240 that are closest to apex 230 may extend through channel 222. Two tapered surfaces 232 of wall 226 may extend (be angled) away from apex 230. Tapered surfaces 232 may transition to two side surfaces 234, which may terminate in opening 224. Side surfaces 234 may each be approximately perpendicular to central longitudinal axis 210 (approximately parallel to a diameter of a cross-section of link 220). Side surfaces 234 may extend approximately halfway through link 220, such that each of side surfaces 234 may begin approximately along a same diameter of a cross-section of link 220.

Apex 230, tapered surfaces 232, and side surfaces 234 may collectively define a perimeter of channel 222. Side surfaces 234 may terminate at a radially outer edge of wall 226. A gap between radially outer ends of side surfaces 234 may define opening 224. Opening 224 may extend approximately parallel with central longitudinal axis 210. As shown in FIG. 4, channel 222 may have a shape that is similar to a square or rectangle (formed by side surfaces 234) combined with an adjacent rounded triangle (formed by tapered surfaces 232 and apex 230). In other words, channel 222 may have an approximately pentagon or rounded pentagon shape. A width of channel 222 may be between lumens 240, thus allowing for a portion of wall 226 forming lumens 240 to be sufficiently resilient during bending. As discussed above, apex 230 may be complementary to a shape of cable 214. Tube 212 may be received within a portion of channel 222 defined by side surfaces 234 and/or tapered surfaces 232. Tube 212 may extend through channel 222 between cable 214 and opening 224. In some aspects, a tapered shape of channel 222 as it nears apex 230 may inhibit tube 212 from moving closer to apex 230 than desired (a diameter of tube 212 may retain tube 212 in larger portions of channel 222).

It should be understood that one or more of links 220 may alternatively or additionally have different configurations, for example defining different shapes and/or sizes of channel 222 and/or opening 224 defined therethrough. In some implementations, one of links 220 may have a different configuration than another one of links 220, for example defining a variable cross-sectional shape or size of channel 222 defined therethrough (e.g., when assembled).

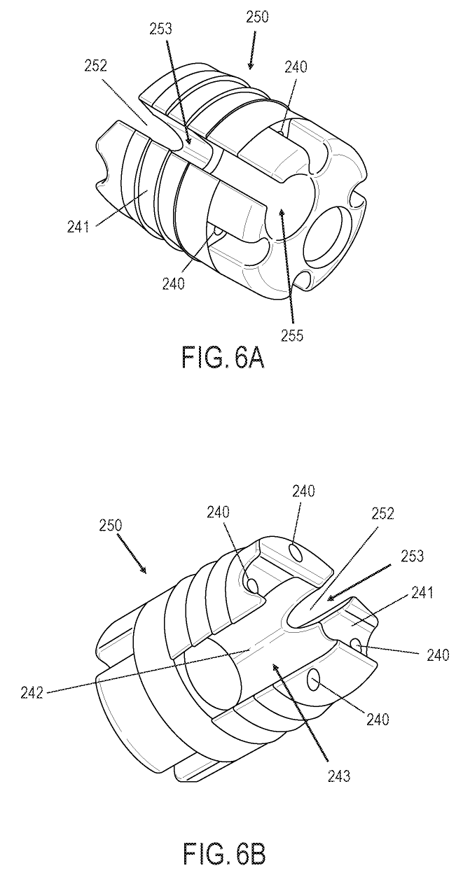

FIG. 5 illustrates a cross-section view of core 250, taken along a plane that includes line A-A. FIGS. 6A and 6B illustrate perspective views of core 250. As shown, core 250 may include a wall 241 defining a first channel 242 and a second channel 252 extending through at least a portion of a length of core 250.For example, first channel 242 and second channel 252 may extend longitudinally through wall 241 approximately parallel to central longitudinal axis 210. Wall 241 may separate first channel 242 from second channel 252, such that first channel 242 and second channel 252 may not be in communication with one another within core 250.

In some examples, as shown in FIG. 5, a proximal portion of first channel 242 may be defined by side surfaces 246 and a curved surface 244 of wall 241. Curved surface 244 may have, for example, a shape of a segment of a circle (e.g., a semicircular shape), an arcuate shape, or another curved shape. In some examples, a radially innermost portion of curved surface 244 may be at or near central longitudinal axis 210. Side surfaces 246 may extend approximately perpendicularly to central longitudinal axis 210 (e.g., approximately parallel to a diameter of the cross-section of core 250). Each of side surfaces 246 may extend from a respective end of curved surface 244 and may extend to a radially outer side of core 250, such that the proximal portion of first channel 242 has an opening 243 on its radially outer side. Opening 243 may be similar to opening 224. For example, opening 243 may be formed by a gap between ends of side surfaces 246 on a radially outer surface of core 250.

A proximal portion of second channel 252, as shown in FIG. 5, may be defined by side surface 256 and an inner surface 254. Inner surface 254 may be curved or may include portions that are straight. Inner surface 254 may be near, and radially outward of, central longitudinal axis 210. As compared with the proximal portion of first channel 242, the proximal portion of second channel 252 may be slightly less deep in a radial direction. Side surfaces 256 may extend approximately perpendicularly to axis 210 (e.g., approximately parallel to a diameter of the cross-section of core 250). Each of side surfaces 256 may extend from a respective end of inner surface 254 and may extend to a radially outer side of core 250, such that the proximal portion of second channel 252 has an opening 253 on its radially outer side. Opening 253 may be similar to opening 224 or opening 243. For example, opening 253 may be formed by a gap between ends of side surfaces 256 on a radially outer surface of core 250.

In some examples, sheath 204 may radially surround portions of core 250 defining first channel 242 and second channel 252, such that first channel 242 and second channel 252 are enclosed via inner surface portions of sheath 204. In other examples, second channel 252 may have another wall or wall segment enclosing second channel 252, for example that extends between opposing portions of side surfaces 256.

As shown in FIGS. 6A and 6B, second channel 252, including opening 253 may extend along an entire longitudinal length of core 250. Cable 214 may extend through the proximal portion of second channel 252. As shown particularly in FIG. 2B and also in FIG. 6A, a distal portion of second channel 252 may be wider than the proximal portion of second channel 252 to accommodate and support imager 216. Second channel 252 may terminate in a rounded distal opening 255 at a distal face of core 250. Opening 253 may facilitate positioning of imager 216 and cable 214. For example, imager 216 may be inserted into core 250 via distal opening 255, and cable 214 may be passed through opening 253.

First channel 242 may also extend through the entire longitudinal length of core 250. However, only a proximal portion of first channel 242 may include opening 243, and a distal portion of first channel 242 may be fully radially surrounded by wall 241, as shown in FIGS. 6A and 6B. Tube 212 may extend through first channel 242. The radially enclosed distal portion of first channel 242 may support tube 212 and may ensure that tube 212 is properly in communication with opening 213. Opening 243 in the proximal portion of first channel 242 may help with positioning of tube 212 within core 250.

As discussed above, cable 214 and tube 212 extend through the same channel 222 within links 220. Channel 222 is in communication with first channel 242 and second channel 252. Within core 250, tube 212 and cable 214 extend through separate channels 242, 252, respectively. Having separate channels in core 250 may facilitate appropriate positioning of cables 214 and tube 212 within distal tip 206. As shown in FIG. 3, opening 243 may be continuous with opening 224, so that tube 212 may smoothly extend through channel 222 and channel 242. Opening 253 may be approximately diametrically opposed to opening 243. A positioning of cable 214 within channel 222 nearer to apex 230 may facilitate cable 214 transitioning into second channel 252.

As shown in FIGS. 6A and 6B, lumens 240 may extend through core 250 and may terminate at a distal face of core 250. Portions of the distal face of core 250 having the distal openings of lumens 240 may be recessed as compared to adjacent portions of the distal face. These recesses may accommodate fixation elements for the articulation wires (e.g., ferrules, crimps, or widened portions).

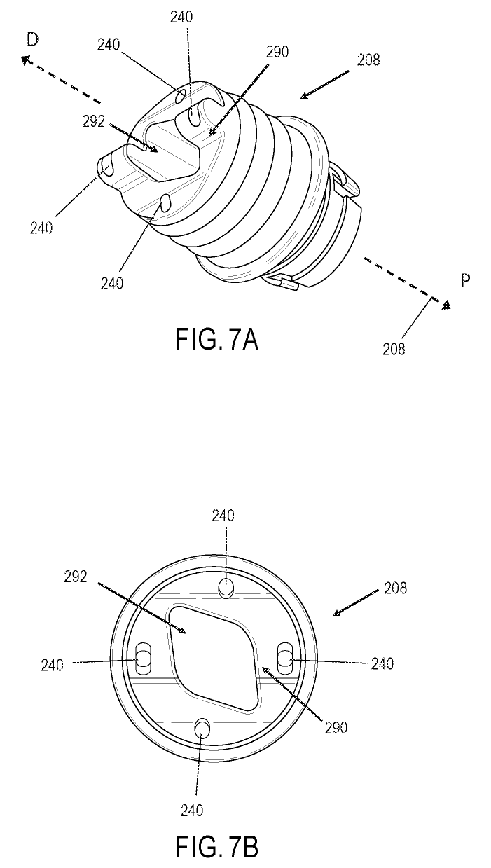

FIG. 7A shows a perspective view of adapter 208 of articulation section 200. FIG. 7B shows a proximally facing view of a distal face of adapter 208. Adapter 208 may include a body having circumferential ridges along an outer periphery and may define a central lumen 292 therethrough. In some examples, as shown in FIG. 7A, central lumen 292 may have a non-circular shape, such as an approximately diamond (or rhombus) or rounded diamond (or rounded rhombus) shaped cross-sectional shape. This cross-sectional shape may allow for a larger area for components to pass through central lumen 292, as compared to a circular shaped cross-section. In aspects, central lumen 292 may be fully radially enclosed (surrounded on all sides by walls of adapter 208).

Elements extending through channel 222 may also extend through central lumen 292. Central lumen 292 may be in communication with channel 222. In some examples, enclosing central lumen 292, for example fully enclosed on all radially outer sides of central lumen 292, may help to transition the elements from the tubular member proximal of adapter 208 to channel 222. Central lumen 292 being fully enclosed may also assist with assembly of a medical device having articulation section 200 by, for example, containing elements such as cable 214 and tube 212 so that they do not fall out of articulation section 200 before it is assembled with more proximal elements.

Adapter 208 may have lumens 240 arranged around the perimeter of central lumen 292, for example arranged symmetrically about central lumen 292 relative to central longitudinal axis 210 defined therethrough. Lumens 240 defined in adapter 208 may align with portions of lumens 240 defined in core 250 and portions of lumens 240 defined in links 220, thereby enabling continuous routing of articulation wires through articulation section 200.

In some examples, adapter 208 may include one or more hinging surfaces 290, for example extending distally away from the body of adapter 208 relative to central longitudinal axis 210. Hinging surfaces 290 may be configured to hingedly interact with a proximal surface of a proximalmost of links 220.

Each of the aforementioned devices, systems, and methods may be used for medical procedures to articulate a distal tip having an articulation section. By providing a medical device capable of articulating the distal tip, known problems associated with endoscopy medical procedures and/or other aspects of invasive surgical procedures may be reduced or avoided. Based on these aspects, physicians or other users of may reduce the overall procedure time, increase efficiency of procedures, and/or avoid unnecessary harm to patient anatomy during procedures that involve articulating the distal tip of the medical device.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. An articulation section for a medical device, the articulation section comprising:
a plurality of intermediate links defining a first channel, wherein the first channel has a first opening on a radially outer side of each of the plurality of intermediate links;
a distalmost link defining a second channel in communication with the first channel and a third channel in communication with the first channel, wherein at least a portion of the second channel has a second opening on a radially outer side of the distalmost link, and wherein at least a portion of the third channel has a third opening on a radially outer side of the distalmost link; and
a proximalmost link defining a lumen in communication with the first channel, wherein the lumen is fully enclosed on all radially outer sides of the lumen.

2. The articulation section of claim 1, further comprising a tube positioned within the first channel, the second channel, and the lumen.

3. The articulation section of claim 1, further comprising a cable or a wire positioned within the first channel, the third channel, and the lumen.

4. The articulation section of claim 1, wherein each of the plurality of intermediate links includes a wall defining the first channel, wherein the wall includes an apex, two tapered surfaces extending from the apex, and two side surfaces extending from the two tapered surfaces.

5. The articulation section of claim 4, wherein radially outer ends of the two side surfaces define a gap that defines the first opening.

6. The articulation section of claim 4, further comprising a wire positioned adjacent to the apex.

7. The articulation section of claim 1, wherein the first opening extends along an entire length of a portion of the articulation section having the plurality of intermediate links.

8. The articulation section of claim 1, wherein a cross-sectional shape of the lumen is non-circular.

9. The articulation section of claim 8, wherein the cross-sectional shape of the lumen is a diamond or a rounded diamond.

10. The articulation section of claim 1, further comprising at least one articulation wire extending through a lumen of each extending through the plurality of intermediate links.

11. The articulation section of claim 1, wherein an entire longitudinal length of the third channel has the third opening.

12. The articulation section of claim 1, wherein only a portion of a longitudinal length of the second channel has the second opening.

13. The articulation section of claim 12, wherein only a proximal portion of the second channel has the second opening.

14. The articulation section of claim 1, wherein the third channel has a distal portion that is wider than a proximal portion of the third channel, wherein the distal portion of the third channel includes an imager.

15. The articulation section of claim 1, wherein the first channel has cross-sectional shape that is a pentagon or a rounded pentagon.

16. An articulation section for a medical device, the articulation section comprising:
a plurality of links defining a channel, wherein the channel has an opening on a radially outer side of each of the plurality of links, wherein a wall of each link of the plurality of links defines the channel, wherein the wall includes an apex, two tapered surfaces, and two side surfaces, such that the channel has a pentagonal or a rounded pentagonal shape;
a cable or a wire positioned within the channel near the apex; and
a working channel positioned within the channel between the cable or the wire and the opening.

17. The articulation section of claim 16, wherein the plurality of links are a plurality of intermediate links, wherein the articulation section further comprises a distalmost link having a first channel and a second channel, wherein the working channel extends through the first channel of the distalmost link, and wherein the cable or the wire extends through the second channel of the distalmost link.

18. The articulation section of claim 16, wherein the plurality of links are a plurality of intermediate links and the channel is a first channel, wherein the articulation section further comprises a proximalmost link having a lumen that is enclosed on all radial outer sides of the lumen, and wherein (a) the working channel and (b) the cable or the wire extend through the lumen.

19. An articulation section for a medical device, the articulation section comprising:
a plurality of intermediate links defining a first channel, wherein the first channel has a first opening on a radially outer side of each of the plurality of intermediate links;
a distalmost link defining a second channel in communication with the first channel and a third channel in communication with the first channel, wherein at least a portion of the second channel has a second opening on a radially outer side of the distalmost link, and wherein at least a portion of the third channel has a third opening on a radially outer side of the distalmost link;
a working channel extending through the first channel and the second channel; and
a cable or a wire extending through the first channel and the third channel.

20. The articulation section of claim 19, wherein a distal end of the cable or the wire is coupled to an imager.
